(19) United States
(12) Patent Application Publication
(10) Pub. No.: US 2024/0023929 A1
(43) Pub. Date: Jan. 25, 2024

(54) ULTRASONIC CATHETER

(71) Applicant: SoundCath, Inc., Canoga Park, CA (US)

(72) Inventors: Donald Masters, Sylmar, CA (US); Jesus Andres Lopez, III, Bloomington, CA (US)

(73) Assignee: SoundCath, Inc., Canoga Park, CA (US)

(21) Appl. No.: 17/813,909

(22) Filed: Jul. 20, 2022

(51) Int. Cl. A61B 8/12; A61B 8/00

(52) U.S. Cl. CPC A61B 8/12; A61B 8/4488; A61B 8/445; A61B 8/463; A61B 8/4494; A61B 2017/00292

(57) ABSTRACT

An ultrasonic catheter is disclosed. The ultrasonic catheter comprises a body having a longitudinal axis and a distal end. A Piezoelectric Micromachined Ultrasonic Transducer (pMUT) array is disposed within the distal end of the body. Further, the pMUT array comprises a plurality of pMUT array elements arranged on a substrate. Further, an insulating material is disposed at the distal end over an imaging window, to provide electrical isolation and transmission of ultrasound signals. The insulating material corresponds to a polyether block amide (PEBA) or Pebax, or thermoplastic elastomer (TPE).

### FIELD OF THE DISCLOSURE

The present disclosure relates generally to the field of ultrasonic catheter. More particularly, some embodiments relate to ultrasonic catheters coated with an electrically insulating material for transmitting and receiving acoustic pulse information.

### BACKGROUND OF THE DISCLOSURE

Use of catheter-based structural and electrophysiological procedures have recently expanded to more complex scenarios, in which an accurate definition of variable individual cardiac anatomy is a key to obtain optimal results. Intracardiac echocardiography (ICE) is a unique imaging modality for high-resolution real-time visualization of cardiac structures, continuous monitoring of catheter location within the heart, and early recognition of procedural complications, such as pericardial effusion or thrombus formation. Further, ICE imaging modality includes additional benefits, such as excellent patient tolerance, reduction of fluoroscopy time, and elimination of need for general anaesthesia or second operator. Currently, ICE imaging modality has largely replaced trans-oesophageal echocardiography as ideal imaging modality for guiding certain procedures, such as atrial septal defect closure and catheter ablation of cardiac arrhythmias, and has an emerging role in others, including mitral valvuloplasty, transcatheter aortic valve replacement, and left atrial appendage closure.

ICE catheters provide cardiologists and heart surgeons with unique viewing perspectives beneficial to the diagnosis and treatment of heart diseases. Typically, ICE involves a single rotating transducer or an array of transducer elements to transmit ultrasound at the tips of the catheters. The same transducers (or separate transducers) are used to receive echoes from the tissue. A signal generated from the echoes is transferred to a console which allows for the processing, storing, display, or manipulation of the ultrasound-related data. ICE catheters are usually used to image the chambers of the heart and surrounding structures. Commercially-available ICE catheters are not designed to be delivered over a guidewire, but instead, have distal ends which can be articulated by a steering mechanism located in a handle at the proximal end of the catheter.

ICE is performed by the primary operator of the interventional procedure under conscious sedation, without the need for endotracheal intubation, and thereby eliminate the risk of oesophageal trauma and other post anaesthesia outcomes. Further, ICE reduces fluoroscopy exposure to both the patient and the operator, may improve outcomes, shortens the procedure time, and facilitates early recognition of complications such as thrombus formation or pericardial effusion. However, ICE catheters when inserted inside the heart, may cause infection to the passage through which ICE catheter is inserted and thereby may create discomfort for the patient. ICE catheters currently in use, does not have a degree of flexibility while making insertion. Further, the patient may be at risk when a transducer array is inserted inside the heart due to electrical impulses supplied. Moreover, ICE catheter currently employed does not provide an acoustic match between the transducer array and bodily fluids the heart. Therefore, there is a need for an improved ultrasonic catheter with a high-density flexible circuit for all transmission and electrical insulated interconnects to enable highly repeatable and stable transmission of signals.

### SUMMARY OF THE DISCLOSURE

By way of introduction, the preferred embodiments described below include an easy-to-use ultrasonic catheter is disclosed. The ultrasonic catheter comprises a body having a longitudinal axis, a proximal end, and a distal end. Further, a Piezoelectric Micromachined Ultrasonic Transducer (pMUT) array is disposed within the distal end of the ultrasonic catheter. The pMUT array is used to scan a patient for generating images to assist a physician. The pMUT array comprises a plurality of pMUT array elements arranged on a substrate. Each of the plurality of pMUT array elements having the pMUT cells activated by beamforming during transmission of ultrasound beams and reception of reflected echoes to form a high-quality ultrasound image. Each of the plurality of pMUT array elements having pMUT cells of multiple diameters, to achieve a wide bandwidth. Further, the pMUT array is electrically isolated from a patient using an insulating material. The insulating material is disposed at the distal end of the ultrasonic catheter over an imaging window, to provide electrical isolation and provide transmission of ultrasound signals. The insulating material corresponds to a polyether block amide (PEBA) or liquid PEBA (for example, available under the trade name PEBAX®) or a thermoplastic elastomer (TPE) (for example, available under the trade name of VISTAMID®). It can be noted that the insulating material dampens transverse waves of the pMUT array, provides a low lose acoustic window, and prevents the distal end of the ultrasonic catheter against electrical leakage to the patient and against electrical break down voltage.

In one embodiment, a method of insulating a distal tip of an ultrasonic catheter, is disclosed. The method comprises dissolving a predefined amount of a polyether block amide (PEBA) in a solvent base. The dissolved PEBA acts to dampen surface lateral waves on a Piezoelectric Micromachined Ultrasonic Transducer (pMUT) array disposed within a distal end of the ultrasonic catheter. In one embodiment, the dissolved PEBA acts as an insulator of the surface lateral waves on a top ground electrode of the pMUT array and creates an ideal acoustic transition from the pMUT array face to liquid blood and other bodily fluids. Further, the method comprises dissolving the PEBA using at least butanol or propanol at a predefined concentration range. Thereafter, the PEBA is casted and cured at the distal end of the ultrasonic catheter over an imaging window. The predefined concentration ranges of the at least butanol or propanol is from 0.01% to 50% by weight. In one embodiment, the predefined concentration ranges of the at least butanol or propanol is from 0.01% to 50% by weight. In another embodiment, the predefined concentration range of propanol is 0.01% to 30% by weight. The PEBA dampens transverse waves of the pMUT array, provides a lubricous surface to allow easy passage through a guide catheter, and prevents the distal end of the ultrasonic catheter against electrical leakage to a patient, and against electrical break down voltage.

In another embodiment, a method of insulating a distal tip of an ultrasonic catheter, is disclosed. The method comprises mixing an insulating material in a solution of propanol at a predefined concentration range and at a predefined temperature. The predefined concentration ranges of the at least butanol or propanol is from 0.01% to 50% by weight. In one embodiment, the predefined concentration ranges of the at least butanol or propanol is from 0.01% to 50% by weight. In another embodiment, the predefined concentration ranges of the solution of propanol is to 30% by weight. In another embodiment, the predefined temperature varies from 122 to 248 degrees Fahrenheit (° F.). Further, the method comprises placing the mixture in a beaker using a stirrer to form an insulating solution. Thereafter, the method further comprises placing the distal tip of the ultrasonic catheter inside the beaker for a predefined time to bring the distal tip to a predefined diameter. In one embodiment, the predefined time varies according to the thickness needed the distal tip of the ultrasonic catheter. The insulating material corresponds to at least one of a polyether block amide (PEBA) or liquid PEBA (for example, available under the trade name PEBAX®), or a thermoplastic elastomer (TPE) (for example, available under the trade name of VISTAMID®).

Other features and aspects of this disclosure will be apparent from the following description and the accompanying drawings.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements, and in which:

FIG. 1 illustrates a perspective view of an ultrasonic catheter, according to an embodiment of the present disclosure;

FIG. 2 illustrates a schematic diagram of the ultrasonic catheter, according to an embodiment of the present disclosure;

FIG. 3 illustrates a distal end of the ultrasonic catheter, according to an embodiment of the present disclosure;

FIGS. 4 illustrates the distal end of the ultrasonic catheter isolated from a patient using polyether block amide (PEBA) or TPE, according to an embodiment of the present disclosure;

FIG. 5 illustrates a sectional view of the ultrasonic catheter having a Piezoelectric Micromachined Ultrasonic Transducer (pMUT) array, according to an embodiment of the present disclosure;

FIG. 6 illustrates a flexible sheath with a marker band to allow location on an X-ray image, according to an embodiment of the present disclosure;

FIG. 7 illustrates the distal end of the ultrasonic catheter with an imaging window bonded to the PEBA to form cover to a catheter shaft, according to an embodiment of the present disclosure;

FIG. 8 illustrates a flowchart showing a method of insulating the distal end of the ultrasonic catheter using the PEBA, according to an embodiment of the present disclosure; and

FIGS. 9-10 illustrate cross sectional images of a heart with the ultrasonic catheter positioned within a right atrium of the heart, according to an embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems, and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

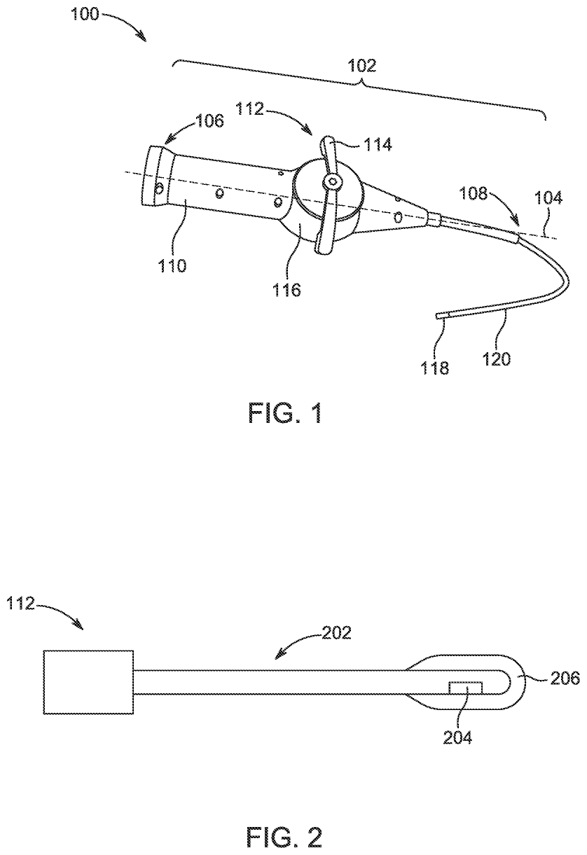

FIG. 1 illustrates a perspective view of an ultrasonic catheter 100, according to an embodiment of the present disclosure. The ultrasonic catheter 100 may comprise a body 102 having a longitudinal axis 104, a proximal end 106, and a distal end 108, a handle assembly 110, a steering control unit 112 having a steering handle 114 and a housing116, and a distal tip 118.

The handle assembly 110 may be positioned between the proximal end 106 and the distal end 108 of the ultrasonic catheter 100. Further, the steering control unit 112 may be positioned within the handle assembly 110. The steering control unit 112 may be provided for articulating the distal tip 118 of the ultrasonic catheter 110. Further, the steering control unit 112 may align face of a Piezoelectric Micromachined Ultrasonic Transducer (pMUT) array (not shown) towards different view including an anterior position and a posterior position inside the heart. In one embodiment, the pMUT array may be used to scan a patient for generating images to assist a physician. As discussed above, the steering control unit 112 may comprise the steering handle 114 and the housing 116 enclosing an actuator (not shown) and a steering hub (not shown). It can be noted that an internal friction occurs between the actuator and the steering hub, and between the actuator and the housing 116, which causes the ultrasonic catheter 100 to retain its adjusted configuration without operator attention. Further, the steering handle 114 may be rotated to facilitate positioning of the distal tip 118 of the ultrasonic catheter 100.

In one embodiment, the steering handle 114 may be rotated to position the distal tip 118 inside a chamber of a heart of a patient. In one embodiment, the steering control unit 112 may comprise a set of steering lines controlled by the steering control unit 112 to articulate bi-directionally a distal segment of the ultrasonic catheter 100 when placed inside the heart. Further, the ultrasonic catheter 100 may be disposed within the chamber of the heart of the patient and coupled to an imaging system (not shown) using a dongle cable (not shown) for displaying two-dimensional (2D) or three-dimensional (3D) images of the chamber of the heart using ultrasound waves and acoustic pulses.

Further, the ultrasonic catheter 100 may be used to perform electrophysiology (EP). The ultrasonic catheter 100 may be used for diagnosis and/or treatment in combination with another imaging modality, such as an x-ray, fluoroscopy, magnetic resonance, computed tomography, or optical system. Both imaging modalities may scan a patient for generating images to assist a physician. The data from the different modalities may be aligned by locating markers with a known spatial relationship to the ultrasound scan in the images of the other modality. In other embodiments, the ultrasonic catheter 100 may be a flexible cylindrical section without the markers and/or without another imaging modality. In one embodiment, the ultrasonic catheter 100 may utilize a microelectromechanical (MEMS) based piezoelectric micro-machined ultrasound transducer (pMUT) or other types of MEMS transducers, interconnected using matched flexible circuits. In one embodiment, the ultrasonic catheter 100 may correspond to an endovascular MEMS ultrasonic catheter utilizing a high-density flexible circuit for all transmission and electrical interconnects. It can be noted that the use of the high-density flexible circuits may enable highly repeatable and stable transmission and return signals. Further, the high density flexible circuit transmission lines may transmit electrical energy from one end to another distal end of the ultrasonic catheter 100.

Further, the ultrasonic catheter 100 may comprise an electronic flex cable 120. The electronic flex cable 120 may be coupled to the handle assembly 110 at one end and to the pMUT array at the other end. The electronic flex cable 120 may be referred to as a flexible cable. It can be noted that the electronic flex cable 120 may be bend or tilt towards the anterior position and/or posterior position inside the chamber of the heart. In one embodiment, the electronic flex cable 120 may comprise a strand, wire, and/or thread, and is preferably made from a low profile, durable, non-elastic, and non-conducting material. In one embodiment, steering cables or steering wires may be made from stainless steel. In another embodiment, the steering cables may be made of synthetic materials, such as nylon or similar synthetic fibres, or plastics material, such as urethane, Teflon®, Kynar®, Kevlar®, polyethylene, multi-stranded nylon, or gel-spun polyethylene fibres. For example, the steering cables may be a multi-stranded Spectra® brand nylon line sold as Spiderwire® fishing line (10 lbs. test).

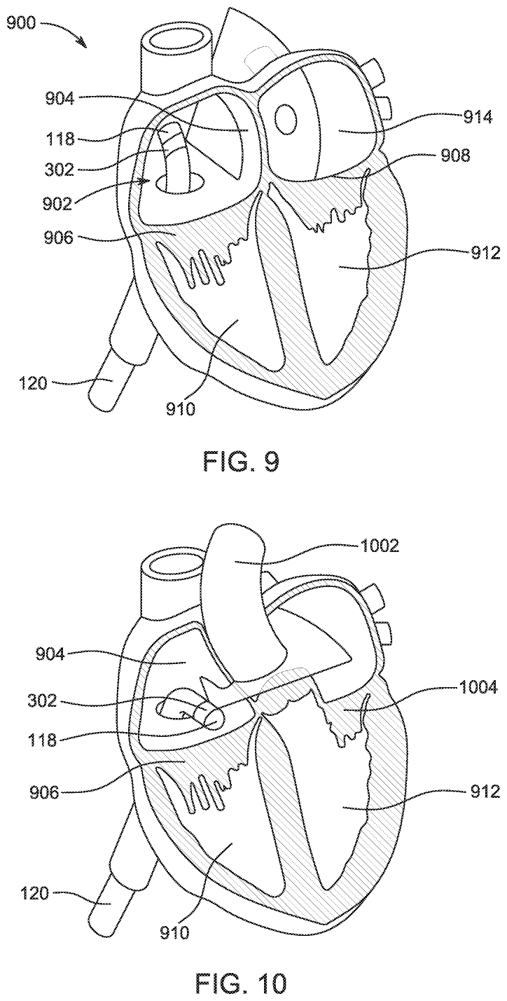

In one embodiment, the ultrasonic catheter 100 may be configured to visualize standard echocardiography views of the heart, such as in a standard version, a right atrium may be visualized. The visualizations performed using the ultrasonic catheter 100 is described in conjunction with FIGS. 9 and 10. The ultrasonic catheter 100 may be employed in transseptal catheterization for several percutaneous interventions, including left heart catheter ablation, atrial septal defect closure for effective alternative to surgical intervention.

FIG. 2 illustrates a schematic diagram of the ultrasonic catheter 100, according to an embodiment of the present disclosure. FIG. 2 is described in conjunction with FIG. 1.

The ultrasonic catheter 100 may be provided to transmit ultrasound signals inside the chamber of the heart of the patient. As discussed above, the ultrasonic catheter 100 may comprise the body 102 having the longitudinal axis 104, the proximal end 106, and the distal end 108. The distal end 108 of the body 102 may be coupled with an electrically isolated shaft 202. In one embodiment, the ultrasonic catheter 100 may be a flexible elongate member with the body 102. Further, the body 102 may comprise a Piezoelectric Micromachined Ultrasonic Transducer (pMUT) array 204 disposed within the distal end 108 of the ultrasonic catheter 100. The pMUT array 204 may be enclosed within the distal tip 118 of the ultrasonic catheter 100. Further, the distal tip 118 of the ultrasonic catheter 100 may be coated with an insulating material 206. It can be noted that the insulating material 206 may correspond to liquid PEBA.

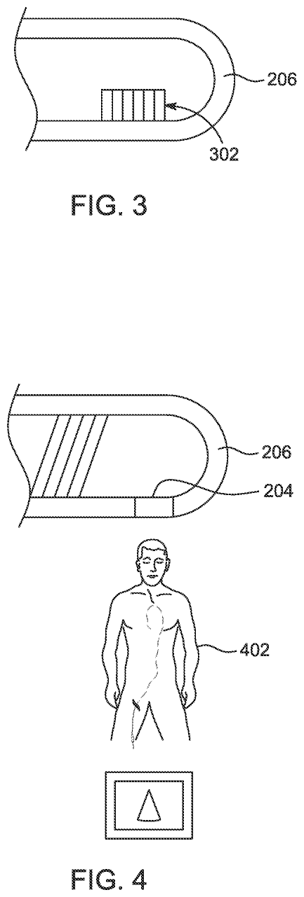

Further, the insulating material 206 may be disposed at the distal end 108 over an imaging window 302, as shown in FIG. 3, to prevent electrical breakdown and leakage of electrical signals from the ultrasonic catheter 100. The insulating material 206 may be a copolymer material such as, but not limited to, polyether block amide (PEBA, for example, available under the trade name PEBAX®), and a thermoplastic elastomer (TPE). In one embodiment, the insulating material 206 provides a low loss acoustic window. It can be noted that the insulating material 206 provides a low loss path of acoustic echo when the ultrasonic catheter 100 is inserted inside the heart of a patient 402, as shown in FIG. 4.

In one exemplary embodiment, the insulating material 206 may be made from a polymer material selected from a group of polymers of block polyamide/ethers, polyether block amide (PEBA, for example, available under the trade name PEBAX®), polytetrafluoroethylene (PTFE), ethylene tetrafluoroethylene (ETFE), fluorinated ethylene propylene (FEP), polyoxymethylene (POM), polyether block ester, polyurethane, polypropylene (PP), polyvinylchloride (PVC), polyether-ester, ether or ester based copolymers phthalate and/or other polyester elastomers, polyamide, elastomeric polyamides, ethylene vinyl acetate copolymers (EVA), silicones, polyethylene (PE), Marlex high-density polyethylene, Marlex low-density polyethylene, linear low density polyethylene, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide, polysulfone, nylon, nylon-12, perfluoro(propyl vinyl ether) (PFA), ethylene vinyl alcohol, polyolefin, polystyrene, epoxy, polyvinylidene chloride (PVdC), poly(styrene-b-isobutylene-b-styrene), poly carbonates, ionomers, biocompatible polymers, other suitable materials, or mixtures, combinations, copolymers thereof, polymer/metal composites, and the like. In some embodiments the polymeric materials may include a liquid crystal polymer (LCP). It can also be noted that the insulating material 206, preferably, PEBA (for example, available under the trade name PEBAX®) , or TPE may dampen surface lateral waves on an pMUT array 204 or transverse waves of the pMUT array 204. In one embodiment, the insulating material 206 may provide an acoustic match between the pMUT array 204 and the bodily fluid inside the chamber of the heart. Further, the insulating material 206 may provide a strong bond between the imaging window 302 and the distal end 108 of the body 102 of the ultrasonic catheter 100. In one embodiment, the ultrasonic catheter 100 may be directly connected to an imaging system ground (not shown).

Further, the electrically isolated shaft 202 may be coated with the insulating material 206 past the imaging window 302 towards the distal end 108 of the ultrasonic catheter 100. In one embodiment, the insulating material 206 may be a copolymer material. In one embodiment, the insulating material 206 may be a liquid PEBA solution coated past the imaging window 302 towards the distal end 108 of the ultrasonic catheter 100. The imaging window 302 may allow ultrasound beams to pass back and forth to the pMUT array 204. In one embodiment, the imaging window 302 may be tilted towards a view including a posterior view or an anterior view and/or vice versa, using the steering control unit 112.

Further, the distal tip 118 may enclose the pMUT array 204. The distal tip 118 may be coated with the insulating material 206 to provide isolation and transmission of the ultrasound signals. It can be noted that the insulating material 206 may be electrically isolated. Further, the ultrasonic catheter 100 may comprise the electronic flex cable 120 coupled to the handle assembly 110 at one end and to the pMUT array 204 at the other end. The electronic flex cable 120 may be provided to allow tilting the distal tip 118 towards the anterior position and/or posterior position inside the chamber of the heart.

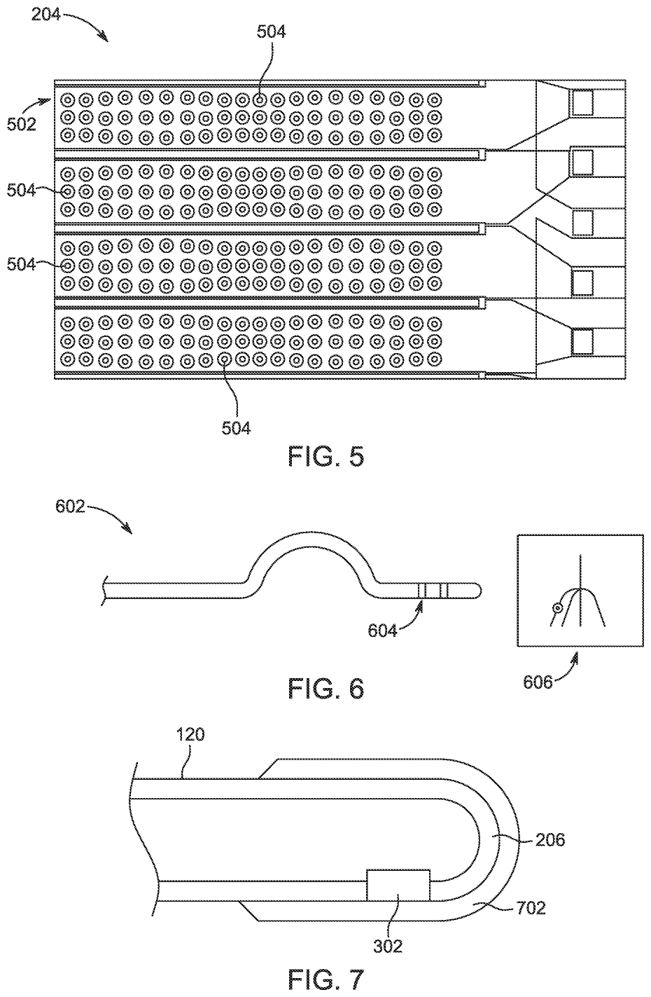

Referring to FIG. 5, a sectional view of the ultrasonic catheter 100 having the pMUT array 204 with a plurality of pMUT array elements 502, is disclosed, according to an embodiment of the present disclosure. Further, each of the plurality of pMUT array elements 502 may have a plurality of individual pMUT cells 504 arranged in a manner to provide a wide bandwidth of the individual focussed beam. In one embodiment, each of the plurality of individual pMUT cells 504 may have a different diameter from each other. It can be noted that the different diameters of the plurality of individual pMUT cells 504 achieve a wide bandwidth beam. It can also be noted that tightly control axial beam may be achieved by the insulating material 206 which dampens the transverse waves of the pMUT array 204. In one embodiment, the pMUT array 204 may be constructed from a pMUT array containing individual elements of different diameters. In one embodiment, to achieve wider bandwidth with pMUT arrays, multiple diameters of pMUT cells may be integrated into one element. It can be noted that by arranging pre-shaped pMUTs with different diameters, a broader bandwidth and improved sensitivity can be realized through the complex interaction between the individual pMUT elements. In one embodiment, the plurality of pMUT array elements 502 of the pMUT array 204 may have multicable diameter cells. For example, in 3 elements, there are 5 different dome diameters, and each array is of a different size, such as 300 μm. Each of the plurality of pMUT array elements 502 having the pMUT cells activated by beamforming during transmission of ultrasound beams and reception of reflected echoes to form a high-quality ultrasound image. Each of the plurality of pMUT array elements 502 having the pMUT cells of multiple diameters, to achieve a wide bandwidth.

Further, the pMUT array 204 may correspond to pMUT and the plurality of pMUT array elements 502 may correspond to a plurality of pMUT elements. In one embodiment, the plurality of pMUT elements may be directed to transmit and receive, the ultrasound beams having the bandwidth including the predetermined fundamental mode vibration of each of the plurality of pMUT elements, such that a single pMUT element can transmit and receive multiple fundamental mode vibrations simultaneously. Further, the electronic flex cable 120 receives the at least one signal from the plurality of pMUT elements. It can be noted that the at least one signal may correspond to the at least one ultrasound beam. The at least one signal may be transmitted to the imaging device for further processing. It can be noted that the plurality of pMUT elements may be used to create the individual focussed beam.

In one alternate embodiment, the pMUT array 204 may include a cover portion that presents a circular cross-section. It can be noted that a feature of pMUT array 204 is typical in ultrasonic imaging catheters. Due to the severe space restrictions imposed by the small diameter of intracardiac catheters, the pMUT array 204 is typically limited to a linear phased array made up of several individual transducer elements, such as 64 transducers or elements. The transducers have a flat surface from which sound is omitted and echoed sound is received. As is well known in the art, the individual transducer elements are pulsed by an ultrasound control system so that the emitted sound waves are constructively combined into a primary beam. By varying the time at which each transducer element is pulsed, an imaging system may render the individual beams into a focussed image which can be swept through an arc in order to obtain the 2D image. As a result, the pMUT array 204 emits ultrasound along a plane that is perpendicular to the face of the transducer arrays. Thus, the pMUT array 204 emits sound along a plane that is perpendicular to the assembly. In one embodiment, when the pMUT array 204 is rotated about a pivot head (not shown) to 90° from the ultrasonic catheter 100 centreline, the plane of the 2D image is orthogonal to the plane of the 2D ultrasound image that will be generated when the ultrasonic imaging array is positioned at a zero angle of rotation.

Referring to FIG. 6, the ultrasonic catheter 100 may comprise a flexible sheath 602 with a marker band 604 to allow location on an X-ray image 606, according to an embodiment of the present disclosure. The flexible sheath 602 may have the marker band 604 towards the distal end 108 or the distal tip 118 of the ultrasonic catheter 100, to allow a passage into the chamber of the heart of the patient and thereby allow location on the X-ray image 606. It can be noted that the distal tip 118 of the ultrasonic catheter 100 may be coated with the insulating material 206 to provide electrical isolation and transmission of ultrasonic signals generated by the ultrasonic catheter 100. In one embodiment, the flexible sheath 602 may be inserted inside the chamber of the heart and the marker band 604 may allow location on the X-ray image 606. In one embodiment, the flexible sheath 602 may correspond to the electronic flex cable 120 to allow the passage into the heart and thereby achieve location on the X-ray image 606. In one embodiment, the electrically isolated shaft 202 may be positioned at different angles inside the chamber of the heart to create the individual focussed beam, using the flexible sheath 602.

Referring to FIG. 7, the insulating material 206 may be bonded to the distal tip 118 of the ultrasonic catheter 100, to form a cover layer 702 between the insulating material 206 and the distal tip 118 of the ultrasonic catheter 100. It can be noted that the cover layer 702 may cover up the imaging window 302. It can also be noted that the insulating material 206 may be provided to prevent electrical leakage from the pMUT array 204. In one embodiment, the electrically isolated shaft 202, the pMUT array 204, and the electronic flex cable 120 may be electrically isolated from the patient using the insulating material 206.

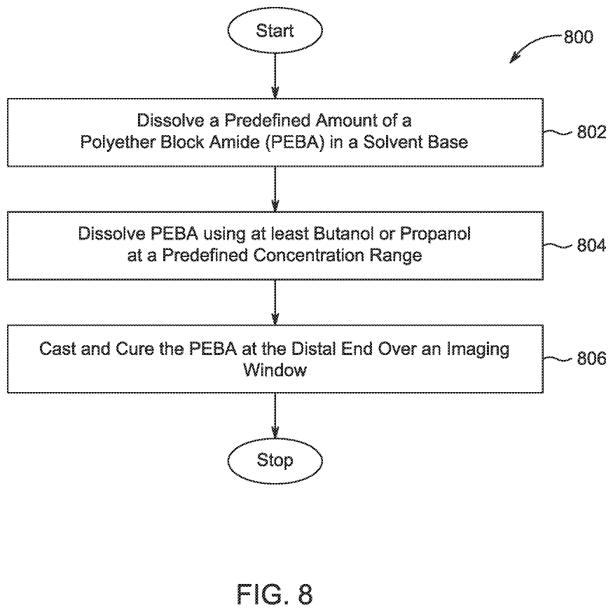

FIG. 8 illustrates a flowchart showing a method 800 of insulating the distal tip 118 of the ultrasonic catheter 100, according to an embodiment of the present disclosure.

At first, a predefined amount of a polyether block amide (PEBA) is dissolved in a solvent base, at step 802. Successively, the PEBA is dissolved using at least butanol or propanol at a predefined concentration range, and at a predefined temperature, at step 804. In one embodiment, the dissolved PEBA acts to dampen the surface lateral waves on a top of the pMUT array 204 and creates an ideal acoustic transition from the pMUT array 204 face to liquid blood and other bodily fluids. In one embodiment, the predefined concentration range of propanol is 0.01% to 30% by weight. In another embodiment, the predefined temperature may vary from 122 to 248 degrees Fahrenheit. In one exemplary embodiment, the predetermined temperature is 130F. Thereafter, the PEBA is casted and cured at the distal end 108 over an imaging window 302, at step 806.

In one exemplary embodiment, a method for insulating the distal tip 118 of the ultrasonic catheter 100 may comprise, at first the insulating material 206 is mixed using a stirrer in a solution of propanol at a predefined concentration range and at a predefined temperature. In one embodiment, the insulating material 206 may correspond to PEBA or Pebax. In one embodiment, the predefined concentration range of propanol is 0.01% to 30% by weight. In another embodiment, the predefined temperature may vary from 122 to 248 degrees Fahrenheit. Finally, the distal tip 118 of the ultrasonic catheter 100 is placed inside the beaker for a predefined time to bring the distal tip 118 o a predefined diamet[user 1]. In one embodiment, the predefined time may vary according to thickness needed for the distal tip 118 or the requirement prescribed by the clinician.

Referring to FIGS. 9-10, cross sectional images of a heart 900 with the ultrasonic catheter

100 positioned within a right atrium 902 of the heart 900, are disclosed. The distal tip 118 of the ultrasonic catheter 100 may be inserted into the right atrium 902 via an inferior vena cava (not shown). In order to perform an adequate imaging of an interatrial septum (IAS) 904 and its neighbouring structures, two standardized views may be used. The movement of the distal tip 118 of the ultrasonic catheter 100 within the right atrium 902 may be controlled by the steering control unit 112. Further, the movement of the distal tip 118 by the steering control unit 112 clockwise or counter clockwise, may allow the imaging window 302 to move from posterior view to anterior view or vice versa.

Further, as shown FIGS. 9-10, to properly place the pMUT array 204 into position for imaging the right atrium 902 and a bicuspid valve 906, the flexible sheath 602 may be introduced into the patient's vascular structure via a femoral vein (not shown). Using a fluoroscopic imaging to monitor the ultrasonic catheter's position, a clinician may advance the distal end 108 of the ultrasonic catheter 100 into the right atrium 902. In order to guide the ultrasonic catheter 100 through turns in the patient's vascular structure, the clinician may rotate the distal tip 118 clockwise or counter clockwise to allow the imaging window 302 to move towards from the anterior position to the posterior position and vice versa. Once the distal tip 118 of the ultrasonic catheter 100 is in the right atrium 902, the clinician may rotate the distal tip 118 so as to introduce an acute bend in the flexible sheath 602 to direct the pMUT array 204 through a tricuspid valve 908 and into a right ventricle 910, as shown in FIG. 9. In this position, a field of view of the pMUT array 204 may include portions of the right ventricle 910, the IAS 904, the bicuspid valve 906, a left ventricle 912, a left atrium 914 and a left ventricular wall. In one embodiment, when the pMUT array 204 is directed clockwise, the right ventricle 910 and right ventricular wall may be imaged. It can be noted that the clinician may twist the ultrasonic catheter 100, while positioned within the heart 900 as illustrated in FIGS. 9-10, the pMUT array 204 may swing about an axis, which could injure the tricuspid valve 908 or cause the pMUT array 204 to strike the IAS 904. FIG. 9 shows a perpendicular short-axis view to visualize interior parts of the IAS 904. The interior parts of the IAS 904 include an aorta 1002 and a mitral valve 1004 towards the left ventricle 912.

In one exemplary embodiment, the standard view is obtained by placing the ultrasonic catheter 100 in a mid—right atrium and the pMUT array 204 in a neutral position facing the tricuspid valve 908. The standard view provides imaging of the right atrium 902, the tricuspid valve 908, the right ventricle 910, and typically an oblique or short-axis view of the aortic valve.

Further, when the ultrasonic catheter 100 may be rotated clockwise the aortic valve in long axis and the right ventricle 910 outflow tract is viewed. In this view, the tricuspid valve 908 that is closer to the pMUT array 204 or the distal tip 118 is a non-coronary cusp, which is in close relationship to a membranous septum and a para-hisian region, whereas the opposite is a right coronary cusp, which is the most anterior of the aortic cusps, directly posterior to the right ventricle 910 outflow tract infundibulum and pulmonic valve. The left ventricle 912 is visualized anterior to the most septal portion of the right atrium 902, and the opening of the coronary sinus becomes evident. In this view, the long axis of the left ventricle 912 outflow tract is identified, and the posterior left ventricle 912 is in view just below the non-coronary cusp.

Further, an additional clockwise rotation of the distal tip 118 allows visualization of the mitral valve 1004 and the IAS 904, with the left atrial appendage anteriorly and the coronary sinus posteriorly. The left atrium 914 appendage is examined for the presence of thrombus at its ostium, and mitral regurgitation may be assessed using a colour Doppler.

In one embodiment, most catheters used in intravascular applications, particularly those with ultrasound transducers, are at least about 10 French in diameter. The electronics and wires needed for ultrasound transducer arrays have made it impractical and expensive to reduce the size of such catheters below about 10 French. Nevertheless, there are benefits in reducing the diameter of the catheter, and technology advances may enable the electronics and control structures to be further reduced in size. The bundling arrangement of the coaxial cables, steering and pivot cables and steering and pivot mechanisms described in more detail below, make it possible to effectively reduce the diameter below about 10 French, to 4, 6, or 8 French or even 3 French (approximately 1 mm).

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

1. An ultrasonic catheter comprising:
a body having a longitudinal axis and a distal end; and
a Piezoelectric Micromachined Ultrasonic Transducer (pMUT) array disposed within the distal end of the body, wherein the pMUT array comprises a plurality of pMUT array elements arranged on a substrate,
wherein an insulating material is disposed at the distal end over an imaging window, to provide electrical isolation.

2. The ultrasonic catheter of claim 1, wherein the pMUT array is electrically isolated from a patient using the insulating material.

3. The ultrasonic catheter of claim 1, wherein each of the plurality of pMUT array elements is a linear phased array.

4. The ultrasonic catheter of claim 1, wherein the distal end of the body is coated with the insulating material to provide transmission of ultrasound signals.

5. The ultrasonic catheter of claim 1, wherein the ultrasonic catheter comprises an electrically isolated shaft coated with the insulating material to cover up a shaft past the imaging window towards the distal end of the body.

6. The ultrasonic catheter of claim 1, wherein the insulating material corresponds to a polyether block amide (PEBA) or Pebax.

7. The ultrasonic catheter of claim 1, wherein the insulating material corresponds to a thermoplastic elastomer (TPE).

8. The ultrasonic catheter of claim 1, wherein the insulating material prevents the distal end of the ultrasonic catheter against electrical leakage to the patient and against electrical break down voltage.

9. The ultrasonic catheter of claim 1, wherein the insulating material dampens transverse waves of the pMUT array.

10. The ultrasonic catheter of claim 1, wherein the insulating material provides a low loss acoustic window.

11. The ultrasonic catheter of claim 1, wherein the pMUT array is used to scan a patient for generating images to assist a physician.

12. The ultrasonic catheter of claim 1, wherein each of the plurality of pMUT array elements having pMUT cells of multiple diameters, to achieve a wide bandwidth.

13. The ultrasonic catheter of claim 12, wherein each of the plurality of pMUT array elements having the pMUT cells activated by beamforming during transmission of ultrasound beams and reception of reflected echoes to form a high-quality ultrasound image.

14. A method of insulating a distal tip of an ultrasonic catheter, the method comprising:
dissolving a predefined amount of a polyether block amide (PEBA) in a solvent base, wherein the dissolved PEBA acts to dampen surface lateral waves on a Piezoelectric Micromachined Ultrasonic Transducer (pMUT) array disposed within a distal end of the ultrasonic catheter;
dissolving the PEBA using at least butanol or propanol at a predefined concentration range; and
casting and curing the PEBA at the distal end over an imaging window.

15. The method of claim 1, wherein the predefined concentration ranges of the at least butanol or propanol is from 0.01% to 50% by weight.

16. The method of claim 1, further comprising an electrically isolated shaft to cover a shaft past the imaging window towards the distal end of the body, wherein the electrically isolated shaft uses the PEBA to cover the shaft up to the imaging window at the distal end of the body.

17. The method of claim 1, wherein the PEBA prevents the distal end of the ultrasonic catheter against electrical leakage to a patient, and against electrical break down voltage.

18. The method of claim 1, wherein the PEBA dampens transverse waves of the pMUT array.

19. The method of claim 1, wherein the PEBA is used with the distal end of the ultrasonic catheter over the imaging window to provide a strong bond of the PEBA and the ultrasonic catheter.

20. The method of claim 1, wherein the PEBA provides a lubricous surface to allow easy passage through a guide catheter.

21. A method of insulating a distal tip of an ultrasonic catheter, the method comprising:
mixing an insulating material in a solution of propanol at a predefined concentration range and at a predefined temperature;
placing the mixture in a beaker using a stirrer to form an insulating solution; and
placing the distal tip of the ultrasonic catheter inside the beaker for a predefined time to bring the distal tip to a predefined diameter.

22. The method of claim 8, wherein the insulating material corresponds to at least one of a polyether block amide (PEBA), Pebax, or a thermoplastic elastomer (TPE).

23. The method of claim 8, wherein the predefined concentration range of propanol is 0.01% to 50% by weight and the predefined temperature varies from 122F to 248F.
